SEQUENCE LISTING (6 sequences)

SEQ ID NO: 1 - DNA, 20 bp, Homo sapiens
acagaagcctgatctgacat

SEQ ID NO: 2 - DNA, 21 bp, Homo sapiens
tgctaacaggtctgggttttg

SEQ ID NO: 3 - DNA, 21 bp, Homo sapiens
tgccaacaacatcaaccacaa

SEQ ID NO: 4 - DNA, 18 bp, Homo sapiens
tccgtcttccgctcctca

SEQ ID NO: 5 - DNA, 21 bp, Homo sapiens
agccgcatcttcttttgcgtc

SEQ ID NO: 6 - DNA, 21 bp, Homo sapiens
tcatatttggcaggtttttct

(19) United States
(12) Patent Application Publication
(10) Pub. No.: US 2009/0148418 A1
(43) Pub. Date: Jun. 11, 2009

(54) Skeletal Site-Specific Characterization of Orofacial and Illiac Crest Human Bone Marrow Stromal Cells in Same Individuals

(75) Inventors: Sunday O. Akintoye, Royersford, PA (US); Songtao Shi, Irvine, CA (US); Pamela G. Robey, Bethesda, MD (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: THE TRUSTEES THE UNIVERSITY OF PENNESYLVANIA, Philadelphia, PA (US)

(21) Appl. No.: 12/086,536

(22) PCT Filed: Dec. 14, 2006
(86) PCT No.: PCT/US2006/048104

§ 371 Date: Dec. 19, 2008

Related U.S. Application Data
(60) Provisional application No. 60/751,078, filed on Dec. 15, 2005.

(51) Int. Cl. A61K 35/12; C12Q 1/02; G01N 33/53

(52) U.S. Cl. 424/93.7; 435/29; 435/7.2

(57) ABSTRACT

The present invention relates to methods and compositions useful for the detection, enrichment, and use of orofacial-derived bone marrow stromal cells. In one aspect, the invention relates to the use of characteristics specific to orofacial-derived bone marrow stromal cells to distinguish orofacial-derived bone marrow stromal cells from other cells.

### CROSS-REFERENCE TO RELATED APPLICATIONS

This application is the U.S. national phase application filed under 35 U.S.C. § 371 claiming benefit to International Patent Application No. PCT/US2006/048104, filed on Dec. 14, 2006 and U.S. Provisional Patent Application No. 60/751,078, filed on Dec. 15, 2005, which is entitled to priority under 35 U.S.C. § 119(a) each of which application is hereby incorporated herein by reference in its entirety.

### BACKGROUND OF THE INVENTION

Autologous bone grafts used to stimulate new bone formation at sites of orofacial osseous defects are commonly obtained from several donor sites including orofacial, axial and appendicular bones. Bridging orofacial defects with grafts obtained from an orofacial donor site are usually more successful than those from non-orofacial sites, indicating anatomic skeletal site-specific differences affect graft integration [18, 30, 37]. Added evidence that orofacial bone development differs from that of axial and appendicular bone formation is suggested by the existence of skeletal diseases such as cherubism [42] and hyperparathyroid jaw tumor syndrome [41], which affect only jaw bones. In addition, craniofacial fibrous dysplasia is histologically and radiologically distinct from fibrous dysplasia in axial and appendicular bones [1,35]. The existence of site-specific variation in bone cell responses has been suggested based on skeletal sitedependent differences in the production of Insulin-like Growth Factor (IGF) system components by cultured human bone cells at various skeletal sites [24]. It is also noteworthy that the basic anatomy of axial and appendicular skeletons has been preserved despite habitat-specific adaptation, in sharp contrast to the craniofacial skeleton which has passed through different morphological modifications. Embryological development and amalgamations of the complex array of bones and cartilage in the craniofacial skeleton indicate that molecular mechanisms controlling skeletogenesis in the head are unique and different from those occurring in other body sites [17]. The cranial vault has a dual neural crest and mesodermal origin; the maxilla, mandible including alveolar bone, dentine, pulp and periodontal ligament are formed exclusively by neural crest cells, while axial and appendicular bones are formed by mesoderm. These clinical, laboratory and developmental differences imply the existence of site-specific properties of progenitor cells in bone marrow.

Within the bone marrow microenvironment are multipotent stromal cells that can differentiate into bone, fat, cartilage, myelosupportive stroma, and perhaps muscle and neural tissues [15, 33, 34]. Isolation and characterization of bone marrow stromal cells have focused on axial and appendicular bones with paucity of information from orofacial sites. Recent reports have identified unique cell populations with multipotent stromal cell properties in dental tissues including dental pulp [14], exfoliated deciduous teeth [27], periodontal ligament [38] and dental extraction socket [26] all with diverse ontogeny and developmental potentials [39].

What is still needed in the art, however, is a way to clearly distinguish bone marrow stromal cells from various skeletal sources within an organism. What is also needed is a way to positively identify an orofacial-derived bone marrow stromal cell, in order to provide, among other things, a source of bone marrow stromal cells useful in the treatment of orofacial bone disorders and deficiencies. The present invention meets these needs.

### SUMMARY OF THE INVENTION

In an embodiment, the invention includes a method of detecting an orofacial-derived bone marrow stromal cell (OF-BMSC), comprising obtaining a first marrow stromal cell from a subject, culturing a first marrow stomal cell, comparing at least one property of a first marrow stromal cell with the corresponding characteristic of a second, non-orofacial marrow stromal cell. A difference between the characteristics of a first and second marrow stromal cell is an indication that the first cell is an OF-BMSC. Differences include at least one of the characteristics selected from the group consisting of positive presence of the STRO-1 marker, increased level of calcium accumulation, more rapid proliferation, delayed senescence, and higher expressed level of alkaline phosphatase, when compared to the characteristics of the second cell.

In an embodiment, the invention includes a method of enriching orofacial-derived bone marrow stromal cells (OF-BMSCs) from a population of bone marrow stromal cells containing at least one OF-BMSC, comprising providing an antibody specific for at least one marker expressed on an OF-BMSC, contacting a population of cells with an antibody under conditions suitable for formation of an antibody-OF-BMSC complex, substantially separating the antibody-OF-BMSC complex from the population of cells and identifying at least one OF-BMSC characteristic selected from the group consisting of increased level of calcium accumulation, rapid proliferation, delayed senescence, and higher expressed level of alkaline phosphatase.

In an embodiment, an OF-BMSC is a human OF-BMSC. In an embodiment, a marker is at least one marker selected from the markers STRO-1, CD-106 and CD-146.

In an embodiment, the invention includes a method of providing an orofacial bone graft to a patient in need thereof, comprising detecting an orofacial marrow stromal cell according to the invention, isolating the orofacial marrow stromal cell, and administering the orofacial marrow stromal cell to said patient.

In an embodiment, the invention includes a method of stimulating new bone formation in an orofacial bone in a patient in need thereof, comprising detecting an orofacial marrow stromal cell according to the invention, isolating an orofacial marrow stromal cell, and administering the orofacial marrow stromal cell to a patient.

In an embodiment, the invention includes a method of providing an orofacial bone graft to a patient in need thereof, comprising administering an isolated orofacial marrow stromal cell to a patient, wherein the orofacial marrow stromal cell becomes engrafted to an orofacial bone in the patient.

In an embodiment, the invention includes a method of stimulating new bone formation in an orofacial bone in a patient in need thereof, comprising administering an isolated orofacial marrow stromal cell to a patient, wherein the orofacial marrow stromal cell stimulates new bone formation in an orofacial bone in the patient.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1E, is a series of images depicting characteristics of orofacial and iliac crest primary human bone marrow stromal cells (hBMSCs). Single cell suspensions of nucleated cells isolated from three skeletal sampling sites (n=4) were plated in 25 cm² plastic flasks for 14 days, fixed and stained with methyl violet. FIG. 1A-1C illustrate fibroblast-like morphology of representative cell monolayers from primary hBMSCs derived from iliac crest (FIG. 1A), maxilla (FIG. 1B) and mandible (FIG. 1C). Macroscopic appearance of isolated colonies formed by mandible primary hBMSCs is illustrated in the lower panel (FIG. 1D) and is also representative of similar colonies formed by maxilla and iliac crest cells. Immunoreactivity of mandible hBMSCs to anti-STRO 1 monoclonal antibody (FIG. 1E) illustrated that 10-20% hBMSCs attached to chamber slides were STRO 1+.

FIG. 2, comprising FIGS. 2A-2D, is a series of images depicting proliferative and life span characteristics of human bone marrow stromal cells (hBMSCs). A comparison of proliferative rates of first passage hBMSCs over 14 days (FIG. 2A) showed that maxilla and mandible cells sustained higher proliferative rate than iliac crest cells within 14 days of culture (n=4; ANOVA, P<0.05, days 5 and 9; P<0.01, day 14). Similarly, maxilla and mandible hBMSCs (OF-MSCs) displayed higher population doublings (FIG. 2B) than iliac crest hBMSCs within 225 days (n=4). Error bars indicate standard deviation. Production of telomerase by PD20 hBMSCs demonstrated by Western blot (FIG. 2C) showed higher telomerase levels in OFMSCs than iliac crest cells (n=4). This was further corroborated by TRAPEZE® assay to evaluate telomerase expression (FIG. 2D).

FIG. 3, comprising FIGS. 3A-3H, is a series of images depicting in vitro osteogenic and adipogenic differentiation of human bone marrow stromal cells (hBMSCs). Culture of hBMSCs with mineralization medium for 6 weeks was followed by Alizarin Red S staining (FIGS. 3A, 3B and 3C). Note that the differences in absorption of the stain by the three cell types are site-specific: iliac crest (FIG. 3A)<maxilla (FIG. 3B)<mandible (FIG. 3C). Maxilla and mandible hBMSCs accumulated more calcium in vitro than iliac crest (FIG. 3D). The differences between iliac crest, maxilla and mandible were statistically significant at P b 0.0001 (n=4; mean of 3-5 experiments). The effect of osteogenic induction on alkaline phosphatase activity of iliac crest, maxilla and mandible hBMSCs is shown in panel (FIG. 3E). All three cell types showed elevated alkaline phosphatase activity 7 days after addition of osteogenic media which was sustained until day 14, but maxilla and mandible hBMSCs (OF-MSCs) were more responsive to induction than iliac crest cells. The differences between iliac crest, maxilla and mandible were statistically significant at P<0.05 respectively (n=4; mean of 3 experiments). Site-specific response to adipogenic induction is shown by Oil Red O staining of hBMSCs cultured for 3 weeks in adipogenic medium. Iliac crest hBMSCs (FIG. 3F) demonstrated larger and more lipid filled cells than maxilla (FIG. 3G) and mandible (FIG. 3H) cells. Arrows point to lipid filled cell clusters (statistical analyses were by one-way ANOVA).

FIG. 4, comprising FIGS. 4A-4F, is a series of images depicting the immunoreactivity of orofacial and iliac crest BMSCs. Iliac crest (left panel), maxilla (middle panel) and mandible (right panel) hBMSCs were attached to 8-well chamber slides and reacted with rabbit antibodies to bone sialoprotein (anti-BSP), top panel (FIGS. 4A-4C), and matrix extracellular phosphoglycoprotein (anti-MEPE), lower panel (FIGS. 4D-4F).

FIG. 5, comprising FIGS. 5A-5L, is a series of images depicting site-specific in vivo bone formation by human bone marrow stromal cells (hBMSCs). Sections of normal bone from iliac crest, maxilla and mandible were compared with in vivo bone formed by transplanted hBMSCs. The first row illustrates microscopic sections of normal iliac crest (FIG. 5A), maxilla (FIG. 5B) and mandible (FIG. 5C) with dense bone and abundant adipose tissue. Note abundant hematopoietic tissue in iliac crest section. The second row illustrates representative sections of bone formed in vivo by noninduced iliac crest hBMSCs (FIG. 5D) and OF-MSCs [maxilla (FIG. 5E) and mandible (FIG. 5F)]. When transplanted hBMSCs were cultured under osteogenic conditions, abundant bone was formed by all three cell types (third row, FIGS. 5G-5I); however, only bone formed by iliac crest hBMSCs contains appreciable amount of hematopoietic tissue (FIG. 5G). The fourth row illustrates a panoramic view (10×) of in vivo bone formation. Note that the bone formed by iliac crest cells hBMSCs (clear arrows, FIG. 5J) was closely packed and contained hematopoietic tissue (HP), while OF-MSCs (maxilla, mandible) formed bone consisting of isolated nodules without hematopoietic tissue (clear arrows, FIG. 5K and FIG. 5L). HP=hematopoiesis; HA=hydroxyapatite/tricalcium phosphate carrier; Oc=osteocyte; FT=fibrous tissue; *=hemorrhage resulting from surgical procedure.

FIG. 6, comprising FIGS. 6A-6B, is a series of images depicting a representation of immunohistochemical staining of in vivo bone with rabbit anti-human osteopontin to determine origin of bone formed. Adjacent 5-elm sections of decalcified paraffin-embedded bone formed by mandible OF-MSCs were stained with hematoxylin/eosin (A) or reacted with rabbit anti-human osteopontin (B). The positive immunoreactivity in panel (B) demonstrated that in vivo bone formed by mandible OF-MSCs was of human origin. Similar results were obtained for iliac crest and maxilla hBMSCs transplants.

FIG. 7, comprising FIGS. 7A-7D, is a series of images depicting site-specific comparisons and quantitative analyses of in vivo bone formation by iliac crest, maxilla and mandible human bone marrow stromal cells (hBMSCs). Bone formation by non-induced iliac crest hBMSCs (dashed lines) was quantifiable at week 6 but limited to bone score b3 during 12 weeks of transplantation (FIG. 7A). However, maxilla and mandible cells formed appreciable bone as early as week 4 and bone score N3 within 12 weeks of transplantation (FIGS. 7B and 7C). When hBMSCs were induced osteogenically before transplantation (solid lines, FIGS. 7A, 7B and 7C), iliac crest and maxilla cells responded by forming more bone, while there was negligible response by mandible cells as displayed by percent change in bone formation (FIG. 7D). The differences in bone formation between the three cell types were statistically significant at P<0.01 (n=4, results were mean of 3 transplantation experiments). Osteogenically induced iliac crest hBMSCs formed appreciable bone as early as week 4 (solid line, FIG. 7A). This indicates that in vivo response of hBMSCs to osteogenic induction is site-specific: iliac crest>maxilla>mandible.

### DETAILED DESCRIPTION OF THE INVENTION

The present invention features methods and compositions related to skeletal site-specific properties of human bone marrow stromal cells (hBMSCs) from orofacial bone. In particular, it is set forth for the first time that hBMSCs from the same individual have skeletal site-specific phenotypic and functional properties when derived from, for example, orofacial bones and axial skeletal bones.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art.

Standard techniques are used for nucleic acid and peptide synthesis. The techniques and procedures are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are those well known and commonly employed in the art. Standard techniques or modifications thereof, are used for chemical syntheses and chemical analyses.

The articles “a” and “an” are used herein to refer to one or to more than one (i.e., to at least one) of the grammatical object of the article. By way of example, “an element” means one element or more than one element.

As used herein, the term “bone marrow stromal cells,” “stromal cells,” “mesenchymal stem cells” or “MSCs” are used interchangeably and refer to the small fraction of cells in bone marrow which can serve as stem cell-like precursors to osteocytes, chondrocytes and adipocytes, and which are isolated from bone marrow by their ability to adhere to plastic dishes. Marrow stromal cells may be derived from any animal. In some embodiments, stromal cells are derived from primates, preferably humans. A human bone marrow stromal cell may be referred to as “hBMSC.”

“Graft” refers to a cell, tissue, organ or otherwise any biological compatible lattice for transplantation.

“Engraft” refers to the process in which a graft begins to grow in a transplanted site, or in which a graft stimulates growth of a cell or tissue at a transplant site, or a combination of the two.

“Immunophenotype” of a cell is used herein to refer to the phenotype of a cell in terms of the surface protein profile of a cell.

“Transplant” refers to a biocompatible lattice or a donor tissue, organ or cell, to be transplanted. An example of a transplant may include but is not limited to a stromal cell, a tissue, a stem cell, a neural stem cell, a skin cell, bone marrow, and solid organs such as heart, pancreas, kidney, lung and liver.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (e.g., growth factors, cytokines, extracellular matrix material, etc.) that facilitate the development of appropriate tissue types and structures.

As used herein, to “alleviate” a disease, disorder or condition means reducing the severity of one or more symptoms of the disease, disorder or condition.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel and anti-parallel strands of a nucleic acid. Thus, a polynucleotide may be either a single-stranded or a double-stranded nucleic acid.

The term “nucleic acid” typically refers to large polynucleotides.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

A “portion” of a polynucleotide means at least at least about twenty sequential nucleotide residues of the polynucleotide. It is understood that a portion of a polynucleotide may include every nucleotide residue of the polynucleotide.

As used herein, the terms “gene” and “recombinant gene” refer to nucleic acid molecules comprising an open reading frame encoding a polypeptide. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins from other species (homologs), which have a nucleotide sequence which differs from that of the human proteins described herein are within the scope of the invention. Nucleic acid molecules corresponding to natural allelic variants and homologs of a cDNA of the invention can be isolated based on their identity to human nucleic acid molecules using the human cDNAs, or a portion thereof, as a hybridization probe according to standard hybridization techniques under stringent hybridization conditions.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

A “recombinant polypeptide” is one which is produced upon expression of a recombinant polynucleotide.

By the term “specifically binds,” as used herein, is meant a compound, e.g., a protein, a nucleic acid, an antibody, and the like, which recognizes and binds a specific molecule, but does not substantially recognize or bind other molecules in a sample.

As used herein, to “treat” means reducing the frequency with which symptoms of a disease, disorder, or adverse condition, and the like, are experienced by a patient.

“Enriching,” as the term is used herein, refers to the process by which the concentration, number, or activity of something is increased from a prior state. For example, a population of 100 spermatogonial stromal cells is considered to be “enriched” in spermatogonial stromal cells if the population previously contained only 50 spermatogonial stromal cells. Similarly, a population of 100 spermatogonial stromal cells is also considered to be “enriched” in spermatogonial stromal cells if the population previously contained 99 spermatogonial stromal cells. Likewise, a population of 100 spermatogonial stromal cells is also considered to be “enriched” in spermatogonial stromal cells even if the population previously contained zero spermatogonial stromal cells.

As the term is used herein, “population” refers to two or more cells.

As the term is used herein, “substantially separated from” or “substantially separating” refers to the characteristic of a population of first substances being removed from the proximity of a population of second substances, wherein the population of first substances is not necessarily devoid of the second substance, and the population of second substances is not necessarily devoid of the first substance. However, a population of first substances that is “substantially separated from” a population of second substances has a measurably lower content of second substances as compared to the non-separated mixture of first and second substances.

In one aspect, a first substance is substantially separated from a second substance if the ratio of the concentration of the first substance to the concentration of the second substance is greater than about 1. In another aspect, a first substance is substantially separated from a second substance if the ratio of the concentration of the first substance to the concentration of the second substance is greater than about 2. In yet another aspect, a first substance is substantially separated from a second substance if the ratio of the concentration of the first substance to the concentration of the second substance is greater than about 5. In another aspect, a first substance is substantially separated from a second substance if the ratio of the concentration of the first substance to the concentration of the second substance is greater than about 100. In still another aspect, a first substance is substantially separated from a second substance if the ratio of the concentration of the first substance to the concentration of the second substance is greater than about 50. In another aspect, a first substance is substantially separated from a second substance if the ratio of the concentration of the first substance to the concentration of the second substance is greater than about 100. In still another aspect, a first substance is substantially separated from a second substance if there is no detectable level of the second substance in the composition containing the first substance.

“Substantially homogeneous,” as the term is used herein, refers to a population of a substance that is comprised primarily of that substance, and one in which impurities have been minimized.

“Maintenance” of a cell or a population of cells refers to the condition in which a living cell or living cell population is neither increasing or decreasing in total number of cells in a culture. Alternatively, “proliferation” of a cell or population of cells, as the term is used herein, refers to the condition in which the number of living cells increases as a function of time with respect to the original number of cells in the culture.

A “defined culture medium” as the term is used herein refers to a cell culture medium with a known composition.

A molecule (e.g., a ligand, a receptor, an antibody, and the like) “specifically binds with” or “is specifically immunoreactive with” another molecule where it binds preferentially with the compound and does not bind in a significant amount to other compounds present in the sample.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As the term is used herein, a cell is said to be “eliminated” from a population of cells, or from a culture medium, when the cell no longer exerts one or more of a physical, biological or chemical effect on the population of cells or culture medium. For example, a cell may be eliminated from a culture medium by physically removing the cell using FACS or by using an antibody specific for a cell surface marker unique to that cell. A cell may also be eliminated from a culture medium by rendering the biological activity of that cell inert, such as, for example, by using a neutralizing antibody that is specific for that cell.

A cell is “essentially eliminated” from a population of cells, or from a culture medium, when most, but not all of the total number of such cells no longer exerts one or more of a physical, biological or chemical effect on the population of cells or culture medium. For example, a particular type of cell may be essentially eliminated from a culture medium if at least 75% of the cells of that type are removed from the culture medium by using an antibody specific for a cell surface marker unique to that cell. More preferably, at least 80% of the cells are eliminated from the culture medium, even more preferably, at least 85%, more preferably, at least 90%, and even more preferably, at least 95% of the cells are eliminated from the culture medium.

### DESCRIPTION OF THE INVENTION

### A. Methods of Detecting and Enriching Orofacial Marrow Stromal Cells

OF-BMSC according to the invention have characteristics which enable the detection, enrichment, and/or isolation of OF-BMSC. Such characteristics include, but are not limited to, positive presence of the STRO-1 cellular marker, increased level of calcium accumulation, more rapid proliferation, delayed senescence, and higher expressed level of alkaline phosphatase, when compared to the characteristics of a non-OF-BMSC. Further, OF-BMSC are osteogenic, therefore requiring minimal stimulation for use according to the invention.

The present invention features a method of detecting orofacial bone marrow stromal cells (OF-BMSCs). It has been shown for the first time herein that, using characteristics specific for OF-BMSCs, OF-BMSCs can be detected within a population of cells. Orofacial stromal cell detection is useful for various purposes in the field of medical treatment, diagnosis and research, including orofacial stromal-cell based therapies for repopulation of cells in an organism, such as in the maxillary or mandibular region, as well as laboratory research to identify factors responsible for control of the maintenance and proliferation of orofacial stromal cells.

The invention therefore features a method of detecting an orofacial marrow stromal cell, the method comprising the steps of obtaining a first marrow stromal cell from a subject, culturing the marrow stomal cell, and comparing at least one property of said first marrow stromal cell with the corresponding characteristic of a second, non-orofacial marrow stromal cell. A difference between the characteristics of the first and second marrow stromal cells is an indication that the first cell is an orofacial marrow stromal cell. In an aspect of the invention, the difference may be at least one of the characteristics selected from the group consisting of positive presence of the STRO-1 marker, increased level of calcium accumulation, more rapid proliferation, delayed senescence, and higher expressed level of alkaline phosphatase, when compared with a non-orofacial marrow stromal cell.

The present invention also features a method of enriching bone marrow stromal cells (BMSCs). It has been shown herein that, using characteristics specific for OF-BMSCs, OF-BMSCs can be enriched within a population of cells. It has also been shown for the first time herein that, using characteristics specific for OF-BMSCs, OF-BMSCs can be enriched from a population of cells. Stromal cell enrichment is also usefull for various purposes in the field of medical treatment, diagnosis and research, including orofacial stromal-cell based therapies for repopulation of cells in an organism, such as in the maxillary or mandibular region, as well as laboratory research to identify factors responsible for control of the maintenance and proliferation of orofacial stromal cells.

In one embodiment of the invention, a method of enriching BMSCs in a population of marrow-derived cells containing at least one BMSC includes providing an antibody specific for at least one marker expressed on an BMSC, contacting the population of cells with the antibody under conditions suitable for formation of an antibody-BMSCs complex, and substantially separating the antibody-BMSCs complex from the population of cells. In one aspect, the antibody is specific for the STRO-1 marker. In another aspect, the antibody is specific for CD-106. In yet another aspect, the antibody is specific for CD-146.

In another embodiment of the invention, the antibody-isolated cell is further characterized using one or more additional characteristics specific for OF-BMSCs. Such characteristics include, but are not limited to, positive presence of the STRO-1 marker, increased level of calcium accumulation, more rapid proliferation, delayed senescence, and higher expressed level of alkaline phosphatase, when compared with a non-orofacial marrow stromal cell.

In another aspect of the invention, further enrichment of OF-BMSCs is conducted using multiple antibody labeling. That is, at least two antibodies, each specific for a unique marker, can be used to enrich the OF-BMSC.

In one aspect of the invention, an OF-BMSC is a mammalian OF-BMSC. In another aspect, an OF-BMSC is a mouse OF-BMSC. In yet another aspect, an OF-BMSC is a human OF-BMSC.

In one aspect of the invention, OF-BMSCs are derived from one skeletal location. In another aspect, OF-BMSCs are derived from an orofacial skeletal location. In still another aspect, OF-BMSCs are derived from a mandibular orofacial skeletal location. In still another aspect, OF-BMSCs are derived from a maxillary orofacial skeletal location.

The present invention also features a method of enriching OF-BMSCs on the basis of OF-BMSC cell surface markers other than STRO-1. Other markers useful in the present invention include, but are not limited to, CD-106 and CD-146.

As will be understood by the skilled artisan, any marker that can be displayed on an OF-BMSC cell surface can be used in the present invention. In one aspect of the invention, a cell surface marker is a marker that is displayed on the surface of a native OF-BMSC. In another aspect, a cell surface marker is a marker that is displayed on the surface of a cell as a result of manipulation of the cell or the marker. In yet another aspect, a marker is one that has been genetically engineered to be expressed on the cell surface.

As will be understood by the skilled artisan, an OF-BMSC may be genetically manipulated to express a greater or lesser amount of an existing cell surface marker, or may be genetically engineered to express a heterologous protein or an endogenous protein that is not typically displayed on the OF-BMSC cell surface. Techniques and procedures for genetic manipulation of cells to express and display a desired surface marker are generally performed according to conventional methods in the art and various general references (e.g., Sambrook and Russell, 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

In another embodiment of the invention, the marker is treated by chemical modification of a marker typically found on the surface of an OF-BMSC. Chemical modification may include contacting the marker with any protein-modifying agent. As will be understood by the skilled artisan, many protein-modifying agents are known in the art, and it will be apparent to the skilled artisan that a protein-modifying agent useful in the present invention may be altered or created de novo, based on the extensive literature surrounding existing agents.

In another embodiment, a marker is treated by enzymatic action. That is, an OF-BMSC cell surface marker can be treated by contacting the marker an enzyme, such as a protease, that can modify the marker by proteolytic digestion of all or a portion of the marker. Other enzymes useful for modifying a marker include, but are not limited to, enzymes that can add or remove one or more proteinaceous moieties to a marker, enzymes that can add a non-proteinaceous moiety to, remove a non-proteinaceous moiety from, or alter a non-proteinaceous moiety on a marker (eg., glycosyltransferases, lipases), and enzymes that can alter properties of the amino acid subunits of a protein marker, such as stereochemistry-modifying enzymes.

The invention also features a method of detection of an OF-BMSC in a mixed population of marrow-derived cells. As described in detail elsewhere herein, OF-BMSCs can be positively detected within a population of marrow-derived cells by way of the Thy-1 surface antigen, in combination with the identification of one or more OF-BMSC characteristics, including, but not limited to, increased level of calcium accumulation, more rapid proliferation, delayed senescence, and higher expressed level of alkaline phosphatase, when compared with a non-orofacial marrow stromal cell.

### Antibodies

As will be understood by one skilled in the art, any antibody that can recognize and bind to an OF-BMSC marker of interest is useful in the present invention. The cells identified using such an antibody can then be analyzed for one or more additional characteristics of OF-BMSC, as described in greater detail elsewhere herein.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the marker protein is rendered immunogenic (e.g., a marker protein conjugated with keyhole limpet hemocyanin, KLH) and a portion comprising the respective marker protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the marker protein antigens. Rather, the invention should be construed to include other antibodies, as that term is defined elsewhere herein, to OF-BMSC cell surface marker proteins, or portions thereof. Further, the present invention should be construed to encompass antibodies, inter alia, bind to the marker proteins and they are able to bind the marker protein present on Western blots, in solution in enzyme linked immunoassays, in fluorescence activated cells sorting (FACS) assays, in magenetic-activated cell sorting (MACS) assays, and in immunofluorescence microscopy of an OF-BMSC transiently transfected with a nucleic acid encoding at least a portion of the marker protein.

One skilled in the art would appreciate, based upon the disclosure provided herein, that the antibody can specifically bind with any portion of the marker protein and the full-length protein can be used to generate antibodies specific therefor. However, the present invention is not limited to using the full-length protein as an immunogen. Rather, the present invention includes using an immunogenic portion of the protein to produce an antibody that specifically binds with a specific OF-BMSC cell surface marker protein. That is, the invention includes immunizing an animal using an immunogenic portion, or antigenic determinant, of the cell surface marker protein.

The antibodies can be produced by immunizing an animal such as, but not limited to, a rabbit, a mouse or a camel, with a protein of the invention, or a portion thereof, by immunizing an animal using a protein comprising at least a portion of an OF-BMSC cell surface marker protein, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind an OF-BMSC cell surface marker protein.

Once armed with the sequence of a specific OF-BMSC marker and the detailed analysis localizing the various conserved and non-conserved domains of the protein, the skilled artisan would understand, based upon the disclosure provided herein, how to obtain antibodies specific for the various portions of an OF-BMSC marker protein using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an OF-BMSC marker molecule can be used to produce antibodies that are specific only for that marker and do not cross-react non-specifically with other proteins.

The invention encompasses polyclonal, monoclonal, synthetic antibodies, and the like. One skilled in the art would understand, based upon the disclosure provided herein, that the crucial feature of the antibody of the invention is that the antibody bind specifically with an OF-BMSC cell surface marker protein. That is, the antibody of the invention recognizes an OF-BMSC cell or a fragment thereof (e.g., an immunogenic portion or antigenic determinant thereof), on Western blots, in immunostaining of cells, and immunoprecipitates the marker using standard methods well-known in the art.

One skilled in the art would appreciate, based upon the disclosure provided herein, that the antibodies can be used to immunoprecipitate and/or immuno-affinity purify their cognate antigen as described in detail elsewhere herein, and additionally, by using methods well-known in the art. In addition, the antibody can be used to enrich OF-BMSCs in a population of marrow-derived cells. Thus, by using an antibody to an OF-BMSC cell surface marker, OF-BMSCs can be identified, enriched or isolated. One skilled in the art would understand, based upon the disclosure provided herein, that any marker, either native or genetically engineered, expressed on an OF-BMSC cell surface, is thus useful in the present invention.

The skilled artisan would appreciate, based upon the disclosure provided herein, that that present invention includes use of either a single antibody recognizing a single OF-BMSC marker epitope but that the invention is not limited to use of a single antibody. Instead, the invention encompasses use of at least one antibody where the antibodies can be directed to the same or different OF-BMSC marker epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., id., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with OF-BMSC epitopes. The humanized antibodies of the invention have a human framework and have one or more complementarity determining regions (CDRs) from an antibody, typically a mouse antibody, specifically reactive with OF-BMSC. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an OF-BMSC epitope, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to a human OF-BMSC epitope. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1989, Antibodies: A Laboratory Manual, Cold Spring Harbor, N.Y.). Camelid species for the production of antibodies and sundry other uses are available from various sources, including but not limited to, Camello Fataga S. L. (Gran Canaria, Canary Islands) for Old World camelids, and High Acres Llamas (Fredricksburg, Tex.) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example, an OF-BMSC epitope, or fragment thereof, using techniques well known in the art. The whole blood can them be drawn from the camelid and sera can be separated using standard techniques. The sera can then be absorbed onto a Protein G-Sepharose column (Pharmacia, Piscataway, N.J.) and washed with appropriate buffers, for example 20 mM phosphate buffer (pH 7.0). The camelid antibody can then be eluted using a variety of techniques well known in the art, for example 0.15M NaCl, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, N.J.) and eluted using, for example, 0.15M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Pat. Nos. 5,800,988; 5,759,808; 5,840,526, and 6,015,695, which are incorporated herein by reference in their entirety. Briefly, cDNA can be synthesized from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (1989, Molecular Cloning, A Laboratory Manual, Cold Spring Harbor, N.Y.). Methods for purification of mRNA from total cellular or tissue RNA are well known in the art, and include, for example, oligo-T paramagnetic beads. cDNA synthesis can then be obtained from mRNA using mRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (V_{HH}) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (1989, Molecular Cloning, A Laboratory Manual, Cold Spring Harbor, N.Y.).

The clones can be expressed in any type of expression vector known to the skilled artisan. Further, various expression systems can be used to express the V_{HH} peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The V_{HH} immunoglobulin proteins isolated from a camelid species or expressed from nucleic acids encoding such proteins can be used directly in the methods of the present invention, or can be further isolated and/or purified using methods disclosed elsewhere herein.

The present invention is not limited to V_{HH} proteins isolated from camelid species, but also includes V_{HH} proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, V_{H} genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

Once expressed, whole antibodies, dimers derived therefrom, individual light and heavy chains, or other forms of antibodies can be purified according to standard procedures known in the art.

In one embodiment of the invention, a phase antibody library may be generated. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed, such as an OF-BMSC cell surface marker antigen. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CHI) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

### Methods of Isolation of Antibody-OF-BMSC Complexes

Once an antibody is bound to an OF-BMSC cell surface marker, the complex can be isolated. That is, an antibody-bound OF-BMSCs can be substantially separated from a population of marrow-derived cells. Methods of identifying or detecting an antibody-antigen complex are well known in the art, and are described in detail elsewhere herein. The cells isolated using such an antibody can then be analyzed for one or more additional characteristics of OF-BMSC, as described in greater detail elsewhere herein, in order to detect OF-BMSC according to the invention.

Various techniques may be employed to separate the OF-BMSCs containing an antibody-bound cell surface marker from cells that do not have an antibody bound cell surface marker by removing antibody-bound OF-BMSC cells from the cell mixture. Alternatively, various techniques may be employed to separate the OF-BMSCs containing an antibody-bound cell surface marker from cells that do not have an antibody bound cell surface marker by removing from the cell mixture OF-BMSC not bound by an antibody.

In one embodiment, the STRO-1 OF-BMSC cell surface marker is used to separate antibody-bound OF-BMSCs from cells not conjugated with antibody. In one aspect of the invention, the antibodies may be attached to a solid support to allow for crude separation. The separation techniques employed should maximize the retention of viability of the fraction to be collected. For “relatively crude” separations, that is, separations where up to 10%, usually not more than about 5%, preferably not more than about 1%, of the total cells present having the marker, may remain with the cell population to be retained, various techniques of different efficacy may be employed. The particular technique employed will depend upon efficiency of separation, cytotoxicity of the methodology, ease and speed of performance, and necessity for sophisticated equipment and/or technical skill, all of which is within the ability of the ordinary skilled artisan.

Procedures for separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., plate, or other convenient technique. Techniques providing accurate separation include fluorescence activated cell sorters, which can have varying degrees of sophistication, e.g., a plurality of color channels, low angle and obtuse light scattering detecting channels, impedance channels, etc., as well as magnetic activated cell sorters.

Conveniently, the antibodies may be conjugated with markers, such as magnetic beads, which allow for direct separation, biotin, which can be removed with avidin or streptavidin bound to a support, fluorochromes, which can be used with a fluorescence activated cell sorter, or the like, to allow for ease of separation of the particular cell type. Any technique may be employed which is not unduly detrimental to the viability of the remaining cells. Other techniques include, but are not limited to, dense particles for density centrifugation, an adsorption column, an adsorption membrane, and the like.

In one embodiment of the invention, an antibody specific for an OF-BMSC cell surface marker is conjugated to a magnetic bead. A population of marrow-derived cells is contacted with the magnetic bead-antibody conjugate, under conditions suitable for binding of the antibody conjugate to an OF-BMSC displaying the marker. After incubation under conditions suitable for binding, such as, but not limited to, an incubation at 4° C. for 20 minutes, OF-BMSCs positive for the marker are selected by passing the entire sample through a magnetic-based separation apparatus. Upon evacuation of free solution from the apparatus, only the magnetically-retained marker-containing cells will remain. The marker-containing OF-BMSC cells are then eluted from the apparatus, resulting in an enriched or purified population of OF-BMSC cells. In one aspect of the invention, an OF-BMSC marker is STRO-1.

After substantial enrichment of OF-BMSC cells, generally by at least about 50%, preferably at least about 70%, the cells may now be separated by a fluorescence activated cell sorter or other methodology having high specificity, such as magnetic activated cell sorting. Multi-color analyses may be employed with the FACS which is particularly convenient. The cells may be separated on the basis of the level of staining for the particular antigens. In a first separation, starting with at least about 1×10¹⁰, preferably at least about 3×10¹⁰ cells, an antibody for STRO-1, for example, may be labeled with one fluorochrome, while the antibodies for other OF-BMSC-specific markers may be conjugated to a different fluorochrome.

Fluorochromes which may find use in a multi-color analysis include phycobiliproteins, e.g., phycoerythrin and allophycocyanins, fluorescein, Texas red, and the like. The enriched cells may then be further separated by positively selecting for STRO-1, for example. The method should permit the removal to a residual amount of less than about 20%, preferably less than about 5%, of the non-stromal cell populations.

As set forth above, the enriched cells can then be analyzed for one or more additional characteristics of OF-BMSC, as described in greater detail elsewhere herein, in order to positively detect and enrich OF-BMSC.

### B. Transplantation of OF-BMSCs

In one aspect of the invention, one or more OF-BMSCs can be transplanted into a recipient skeletal donor site. Transplantation methods are generally known in the art, and will not be discussed in extensive detail herein. For general cell transplantation methods, see, e.g., Kuznetsov et al. (J. Bone Miner. Res. (1997) 12:1335-1347).

In an embodiment of the invention, one or more OF-BMSCs are introduced into a skeletal donor site, as described in detail elsewhere herein. For example, a recipient mammal can be anesthetized and the donor site surgically exposed. Donor sites may be prepared by the skilled artisan, based on the guidance set forth in the present invention.

A cell suspension of one or more OF-BMSCs for transplantation can comprise a biocompatible lattice and at least one OF-BMSC at a suitable concentration. Biocompatible lattice materials useful in the present invention include, but are not limited to, spheroidal hydroxyapatite/tricalcium phosphate. The composition and/or particle size of biocompatible lattices useful in the invention can be determined by the skilled artisan, based on the disclosure set forth herein.

By way of a non-limiting example, the transplantation medium is used to culture the cells but is not transplanted with the cells. Upon transplantation, the cell suspension is centrifuged to allow the cells to attach to the carrier while the culture medium is discarded.

The present invention is applicable to any mammal, including humans, as well as non-human transgenic animals.

In an embodiment of the invention, the donor and recipient mammal can be the same mammal. In one aspect, a population of cells comprising OF-BMSCs are collected from a mammal. OF-BMSCs can be detected in the collected population of cells, and enriched and/or isolated, then administered to the same mammal for therapeutic treatment as required. That is, OF-BMSCs can be derived from an orofacial skeletal marrow source in a mammal, and delivered to another orofacial site in the same mammal, for a therapeutic purpose. As set forth elsewhere herein, the advantages of administering OF-BMSCs to an orofacial skeletal donor site include, but are not limited to, increased success of BMSC transplantation, site-specific tissue regeneration, and faster recovery of recipients in need of site-specific orofacial skeletal marrow stromal cell therapy.

A recipient in need of therapy according to the present invention may include, among others, a patient in need of an orofacial bone graft, a patient in need of an orofacial stromal cell transplantation, and a patient requiring orofacial site-specific stimulation of new bone formation. Such patients and conditions include, but are not limited to, patients requiring improvement of osseous implants, patients requiring healing of osteonecrosis, and patients requiring healing of tooth extraction sockets.

In another embodiment of the invention, the donor and recipient mammal can be two separate mammals. In one aspect, a population of cells comprising OF-BMSCs are collected from a first mammal. OF-BMSCs can be detected in the collected population of cells, and enriched and/or isolated, then administered to a second mammal for therapeutic treatment as required. That is, OF-BMSCs can be derived from an orofacial skeletal marrow source in a mammal, and delivered to an orofacial site in another mammal, for a therapeutic purpose.

In one aspect, a donor mammal and a recipient mammal are different species. By way of a non-limiting example, OF-BMSCs may be obtained from a human donor and administered to an orofacial skeletal site in a murine recipient.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Materials and Methods

### Samples and Cell Culture

Trabecular bone isolated with a ronguer from four 3rd molar surgical sites (maxilla and mandible) and simultaneous iliac crest marrow aspirates were obtained from 7 normal volunteers (ages 18-27 years, 5 males, 2 females). Patients gave informed consent to participate in study protocols approved by the Institutional Review Board of the National Institutes of Health. Samples were obtained from the three skeletal sites in all 7 study subjects. Primary hBMSCs were established in α-modified Minimum Essential Medium (α-MEM) (Invitrogen, Life Technologies, Carlsbad, Calif.) by modifying a previously described method [21].

Briefly, maxilla and mandible trabecular bone samples were gently rinsed several times with α-MEM to eliminate residual oral fluids. Marrow contents were released with a #15 surgical blade into culture dishes containing α-MEM. Iliac crest marrow aspirates were centrifuged for 10 min at 135×g, and the cell pellets were re-suspended in α-MEM. Marrow cells from all three sampling sites were processed separately, but similarly, by repeated pipetting and passage through 16G and 20G needles to disperse the cells followed by subsequent filtration through a 70 μm nylon cell strainer (BD Bioscience, San Jose, Calif.). To establish multi-colony-derived strains, nucleated cells (maxilla=1.3×104 cells/cm2, mandible=1.3×104 cells/cm2 and iliac crest=4.7×104 cells/cm2) were cultured in 25 cm² plastic flasks containing growth medium: α-MEM supplemented with 20% fetal bovine serum (Equitech Bio Inc, Kerville, Tex.), 100 U/ml penicillin, 100 mg/ml streptomycin sulfate and 2 mM glutamine (BioSource International Camarillo, Calif.). Osteogenically induced hBMSCs were established using osteogenic growth medium containing supplements of 10⁻⁸ M dexamethasone (Sigma-Aldrich, St. Louis, Mo.) and 100 μM L-ascorbic acid phosphate magnesium-hydrate (Wako Chemicals, Richmond Va.). For these experiments, nucleated cells were plated in separate 25 cm² flasks using maxilla (0.7×10⁴ cells/cm²), mandible (0.7×10⁴ cells/cm²) and iliac crest (2.3×10⁴ cells/cm²). Plating cell density was higher for iliac crest cells due to the presence of a greater quantity of peripheral blood cells. Cells were incubated at 37° C., in a humidified atmosphere of 5% CO₂ and air. Growth medium was changed on day 1 for aspirate and day 7 for maxilla and mandible hBMSC cultures and once a week thereafter until 75% confluence. Subconfluent primary hBMSCs were released with trypsin-EDTA (Invitrogen, Life Technologies, Carlsbad, Calif.) and further characterized. For all experimental methods described below, maxilla, mandible and iliac crest samples from each subject were compared simultaneously.

### Colony Forming Efficiency Assay

Nucleated cells were cultured in triplicate 25 cm² plastic culture flasks at 103, 104 and 105 cells/flask with non-osteogenic growth medium [20]. Cells were fixed on day 10 with 100% methanol, stained with methyl violet (Sigma-Aldrich, St. Louis, Mo.) and aggregates of 50 or more cells were counted as colonies.

### Cell Proliferation

The proliferation rate of first passage hBMSCs was assessed by growth curve analyses [10,36] after seeding 1×10⁴ cells/well in triplicate 6-well plates (Corning Life Sciences, Acton, Mass.) containing growth medium with and without osteogenic inducers. Cells were released on days 1, 3, 5, 7, 9 and 14 and counted with a particle and cell size analyzer (Z1™ Coulter Counter®, Beckman-Coulter, Inc. Fullerton, Calif.) to plot a growth curve.

### Life Span Measurements

The proliferative life span of hBMSCs was assessed by population doublings (PD) calculated from generation number after repeated cell passage at 1:10 split ratio until the cells attained replicative senescence [PD=product of passage number and split ratio] [16,19].

### Telomerase Activity

The presence of human telomerase reverse transcriptase (hTERT) was determined by Western blotting of nuclear extracts isolated with Nuclei EZ Prep®D (Sigma-Aldrich, St. Louis, Mo.) and HeLa cell lysate as control. Nuclear extracts were derived from hBMSCs at two stages: PD20 and PD40, representing population doublings (PD) of 20 and 40. Equal protein amounts (10 μg) of samples and control were tested, using a mouse monoclonal antibody to hTERT (ab5181, Abcam Inc, Cambridge, Mass.) as the primary antibody (1:250 dilution). The expression of telomerase in enzymatically active hBMSC (PD20) from the three sites was assessed by the PCR-based Telomerase Repeat Amplification Protocol using the TRAPEZE® telomerase detection kit (S7700, Chemicon International, Temecula, Calif.) following manufacturer's recommendations.

Briefly, pre-confluent PD20 hBMSCs were trypsinized, washed with PBS (pH 7.3), pelleted and re-suspended at 750 ng/μl in CHAPS lysis buffer supplied in TRAPEZE® kit. Samples tested included extracts and heat inactivated extracts of PD20 hBMSCs, positive telomerase control extract, primer-dimer/PCR contamination control and a quantitation control template. A 50 μl TRAPEZE® master-mix containing 1.5 μg cell extract was incubated at 30° C. for 30 min followed by 2-step PCR at 94° C./30 s and 59° C./30 s for 33 cycles in a Perkin Elmer thermocycler (Perkin Elmer, Boston, Mass.). After amplification, 25 μl of the PCR products was loaded on a 10% non-denaturing polyacrylamide gel electrophoresis and the bands visualized by ethidium bromide staining. As part of the assay internal control, the TRAPEZE® primer mix also included PCR amplification internal control oligonucleotides that produced a band of 36 base pairs in each lane.

### In Vitro Calcium Accumulation

Mineralization potential of hBMSCs was assessed by in vitro calcium accumulation of first passage hBMSCs (1×10⁴ cells/cm²) in duplicate 60 mm dishes (Corning Life Sciences, Acton, Mass.). At confluence, growth medium was changed to mineralization medium (α-MEM, 5% fetal bovine serum, 2 mM glutamine, 100 U/ml penicillin and 100 μg/ml streptomycin, 10⁻⁸ M dexamethasone, 100 μM L-ascorbic-2-phosphate and 5 mM β-glycerophosphate) with medium change 2 times/week [13,22]. At week 6, hBMSCs were fixed with 70% ethanol and stained with 2% Alizarin Red S (pH 4.2). Unbound and nonspecifically bound stain was removed by copious rinsing with distilled water, and calcium-bound stain was extracted with 0.5 N HCl/5% sodium dodecyl sulfate. Absorbance of the extract was determined at 415 nm along with calcium standards (Cat #3605 Sigma-Aldrich, St. Louis, Mo.).

### Alkaline Phosphatase Assay

Alkaline phosphatase production was assayed by a modification of an earlier described method [9]. Briefly, first passage hBMSCs (1×10⁴ cells/cm²) in triplicate 24-well plates (Corning Life Sciences, Acton, Mass.) were cultured for 15 days in α-MEM growth medium with and without osteogenic inducers and change of medium at 2-day intervals. The cell monolayer was lysed at 4° C. on days 3, 7, 9, 11 and 15 with 0.2% cold NP 40 containing 1 mM MgCl₂ and denatured by repeated freeze-thaw on dry ice. Cells were scraped, sonicated three times in water/wet ice mixture and centrifuged at 800×g for 20 min. Supernatants were assayed for total protein [3] and alkaline phosphatase after 30 min incubation at 37° C. using 0.075M p-nitrophenyl phosphate substrate in 0.05 M barbital buffer, pH 9.3. Positive controls (Accutrol™ normal control, A2034, Sigma-Aldrich, St. Louis, Mo.) and standards (LS006130, Worthington Biochemical Corporation, Lakewood, N.J.) were included to plot a standard curve. The reaction was stopped with 0.1 N NaOH and product measured at absorbance of 405 nm.

### Adipogenesis

Adipogenic differentiation was induced as previously described [32]. Briefly, first passage hBMSCs (1×10⁴ cells/cm²) in duplicate 6-well plates (Corning Life Sciences, Acton, Mass.) were cultured with α-MEM growth medium without inducers until confluence before being exposed to adipogenic medium [containing supplements of 10⁻⁸ M dexamethasone, insulin (1 μg/ml), 1-methyl-3-isobutylxanthine (IBMX, 5×10⁻⁸ M) and indomethacin (10⁻⁴ M)] for 3 weeks; medium was changed twice weekly. Similar plates without exposure to adipogenic medium served as control. The cells were fixed with 4% paraformaldehyde, stained with 0.3% Oil Red O and counterstained with 1% Fast green dye. Lipid droplets were identified microscopically. Marrow stromal cell transplantation and in vivo bone formation Bone formation was assessed using the mouse model of in vivo bone formation as previously described [21] in accordance with an institutionally approved animal protocol (NIDCR #02-222). In two separate groups of 3 animals, non-induced and osteogenically induced cells were transplanted. From each skeletal donor site, 2×10⁶ hBMSCs were attached to 40 mg spheroidal hydroxyapatite/tricalcium phosphate (particle size 0.5-1.0 mm, Zimmer, Warsaw, Ind.) and transplanted into 3 separate subcutaneous pockets aseptically created in 8-week-old immunocompromised nude female mice (NIH-III-nu, Charles River Laboratories, Wilmington, Mass.). Transplants were harvested at weeks 4, 6, 10 and 12, fixed in 4% formalin, decalcified in 10% EDTA (pH 8.0) and embedded in paraffin. Five-micrometer sections were deparaffinized, stained with hematoxylin/eosin and bone formation was scored microscopically by two blinded trained observers for semi-quantitative analysis as previously described [21]. The bone scores ranged from 0 (no bone evident within the transplant), 1 (minimal bone evident [1 trabecula]), 2 (weak bone formation occupying only a small portion of the transplant), 3 (moderate bone formation occupying a significant portion but less than 50% of the transplant) and 4 (abundant bone formation, occupying more than 50% of the transplant±hematopoiesis). This bone scoring method has been validated with histomorphometric analysis [25]. The human origin of bone within transplants was established in deparaffinized unstained sections by reaction with primary rabbit anti-human osteopontin polyclonal antibody (Cat # 499265, EMDBiosciences, San Diego, Calif.) followed by enzymatic staining with broad-spectrum immunoperoxidase AEC kit (Zymed Laboratories, San Francisco, Calif.) [6].

### Immunohistochemical Localization of Cell Surface Markers

hBMSCs were subcultured (2×10⁴ cells/well) in 8-chamber slides (Nalge Nunc, Rochester, N.Y.), fixed with 4% paraformaldehyde and non-specific binding was blocked before incubating with primary antibodies. Separate experiments were performed using the following primary antibodies: mouse monoclonal antibody to STRO-1 and rabbit polyclonal antibodies against human bone sialoprotein, endostatin (Chemicon International, Temecula, Calif.), transforming growth factor receptor (TGFβ-R2, Santa Cruz Biotechnology, Santa Cruz, Calif.), fibroblast growth factor receptor 3 (FGF-R3, Novus Biologicals, Littleton, Colo.), vascular endothelial growth factor receptor 1 (VEGF-R1, R&D Systems, Minneapolis Minn.) and matrix extracellular phosphoglycoprotein (MEPE/LF 155). STRO-1 antibody was undiluted, while other antibodies were incubated at dilutions between 1:25 and 1:100 [12, 14, 27, 38]. Positive cells were visualized with enzymatic broad-spectrum immunoperoxidase AEC kit (Zymed Laboratories, San Francisco, Calif.).

### Reverse Transcriptase Polymerase Chain Reaction (RT-PCR)

Total RNA was isolated from second passage maxilla, mandible and iliac crest BMSCs using TRIzol® reagent (Invitrogen Life Technologies, Carlsbad, Calif.). First strand cDNA was prepared with first strand SuperScript™ Double-Stranded cDNA Synthesis Kit (Invitrogen Life Technologies, Carlsbad, Calif.) using an oligo-dT primer. Two microliters of first strand cDNA was added to a total volume of 50 μl PCR buffer containing: 1.5 mM MgCl₂, 200 μM dNTP, 2.5 U Taq DNA polymerase (Promega, Madison, Wis.) and 200 nM of each primer set. Primers sets used were: human endostatin (COL 18A1), GenBank Accession number AF08081, fragment size 240 bp, forward, 5′-ACAGAAGCCTGATCTGACAT-3′ (SEQ ID NO:1); reverse, 5′-TGCTAACAGGTCTGGGTTTTG-3′ (SEQ ID NO:2); human TGFβ-R2, GenBank Accession number M85079, fragment size 255 bp, forward, 5′-TGCCAACAACATCAACCACAA-3′ (SEQ ID NO:3); reverse, 5′-TCCGTCTTCCGCTCCTCA-3′ (SEQ ID NO:4) and human GAPDH, GenBank Accession number M33197, fragment size 816 bp, forward, 5′-AGCCGCATCTTCTTTTGCGTC-3′ (SEQ ID NO:5); reverse, 5′-TCATATTTGGCAGGTTTTTCT-3′ (SEQ ID NO:6). The PCR reaction was carried out in a Perkin Elmer thermal cycler (Perkin Elmer, Boston, Mass.) at 94° C. for 2 min for 1 cycle then 94° C. (1 min), 55° C. (1 min) and 72° C. (1 min) for 35 cycles and final extension at 72° C. (10 min). Fifteen microliters of the amplified products were separated on 2% agarose gel, stained with ethidium bromide and visualized with Kodak ImageStation 440 (Eastman Kodak, Rochester, N.Y.). Bands were analyzed with Scion Image® (Scion Corporation, Frederick, Md.), and abundance of transcript in samples was semi-quantitatively compared with GAPDH expression.

### Statistical Analyses

All experiments were repeated at least three times, each cell type was tested in triplicates, and results were expressed as mean±standard deviation (SD). Differences between iliac crest hBMSCs and OF-MSCs and between noninduced and osteogenically induced cells were compared with one-way analysis of variance (ANOVA) followed by post hoc comparisons with Turkey-Kramer test. In each analysis, differences were considered significant at P<0.05. Inter-observer variability of bone score measurements was assessed with Kappa statistic.

### Example 1

Isolation of hBMSCs from maxilla and mandible samples was limited to small (0.5×0.5 cm) tissue samples that yielded an average of 5×105 nucleated cells per sample. Sample size needed to determine phenotypic differences between orofacial and iliac crest hBMSCs was calculated using preliminary results of in vivo bone formation by osteogenically induced hBMSCs at weeks 10 and 12. In these studies, the mean bone score for iliac crest hBMSCs was 2.18 (standard deviation=0.2), and pooled maxilla and mandible (orofacial) mean bone score was 3.22 (standard deviation=0.04). At a power of 0.8, a level of 0.05 and standard deviation of 0.35 (assuming more variability and equal standard deviation), the sample size needed to determine a difference is 4. However, a total of 7 subjects were enrolled and 21 samples analyzed.

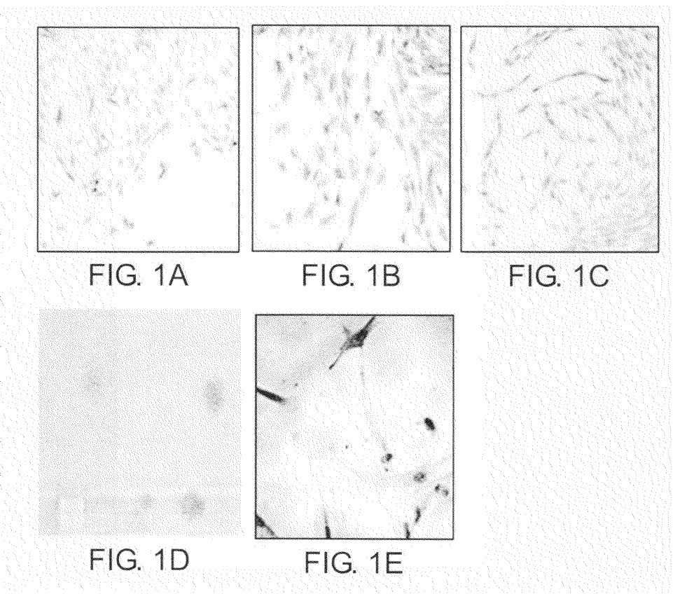

hBMSCs isolated from the three sampling sites formed morphologically variable but discrete colonies with typical fibroblast appearance (FIGS. 1 A, B, C and D). Mean colony forming efficiency (CFE) per 105 nucleated cells, determined using samples from three subjects, was 54, 17 and 37 for maxilla, mandible and iliac crest respectively. The percentage of immunoreactive STRO 1 positive cells (STRO 1+), suggestive of early mesenchymal stromal cells [13,40], ranged between 10 and 20% at all donor sites without appreciable difference between orofacial and axial sites (FIG. 1E). However, the low yield of nucleated cells from orofacial samples restricted the number of samples plated as primary cultures, thus limiting statistical analyses of differences among groups.

### Example 2

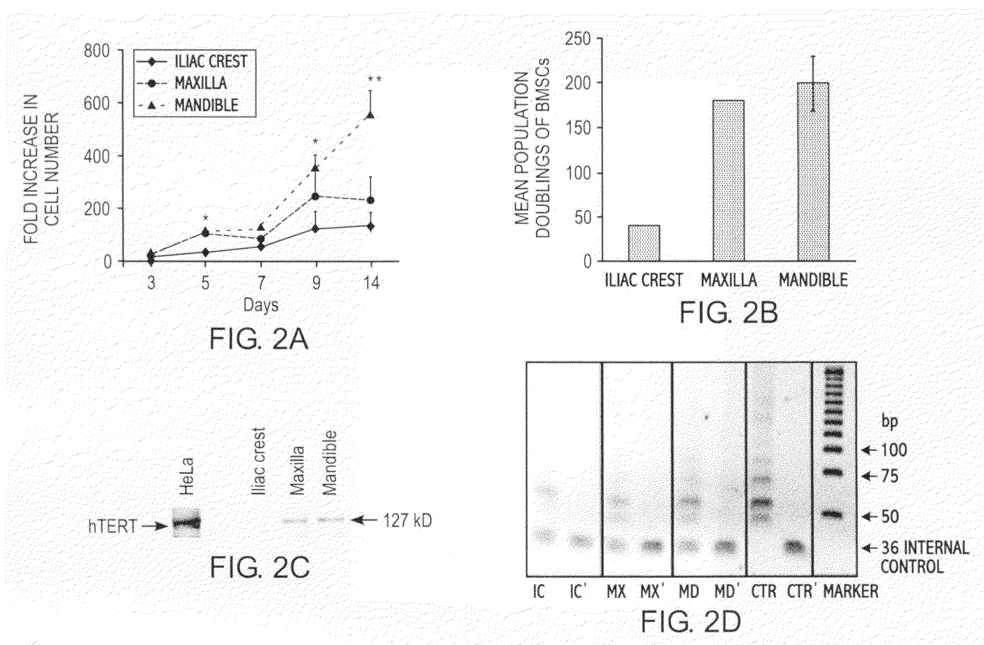

When first passage hBMSCs were plated at low density (103 cells/cm2), significant differences in the proliferative and population doubling (PD) capacities were observed between orofacial (OF-MSCs) and iliac crest hBMSCs. Proliferation rates were much higher in OF-MSCs compared to iliac crest hBMSCs (FIG. 2A), and PDs over 225 days were consistently higher for OF-MSCs than for the iliac-crest-derived cells (FIG. 2B). Average PD sustained by OF-MSCs was PD 180, while iliac crest hBMSCs were limited to PD40 (FIG. 2B). Expression of telomerase by hBMSCs was higher in OF-MSCs than in iliac crest cells by both Western blotting (FIG. 2C) and TRAPEZE® assay (FIG. 2D).

### Example 3

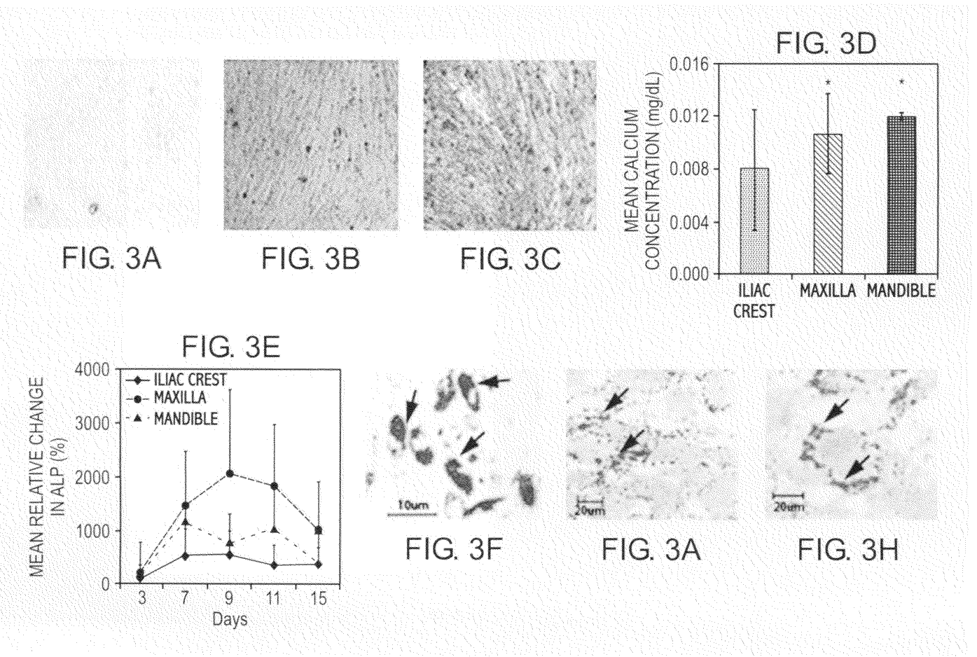

OF-MSCs were more responsive to in vitro osteogenic differentiation than iliac crest hBMSCs as measured by amount of calcium accumulation and alkaline phosphatase activity. Retention of Alizarin Red S stain by OF-MSCs was higher (FIGS. 3A-C). Quantitative analysis of calcium-bound stain (FIG. 3D) by the three cell types cultured for 6 weeks showed significant difference between iliac crest and maxilla (P<0.001) and between iliac crest and mandible (P<0.001) cells. Predictably, the three cell types demonstrated low levels of alkaline phosphatase activity when cultured in nonosteogenic medium, but OF-MSCs produced higher levels of alkaline phosphatase activity than iliac crest hBMSCs in response to osteogenic induction (FIG. 3E). In contrast, differentiation to adipocytes, visualized by Oil Red O staining of lipid-containing cell clusters (FIGS. 3F-H), was more pronounced in iliac crest hBMSCs after 3 weeks of culture in adipogenic medium. The iliac crest lipid clusters were more numerous and larger in size than those of maxilla and mandible hBMSCs. Therefore, in vitro osteogenic differentiation of cultured OF-MSCs, as measured by both calcium accumulation and alkaline phosphatase activity, was more extensive than iliac crest cells, while the iliac crest cells responded better to adipogenic induction.

### Example 4

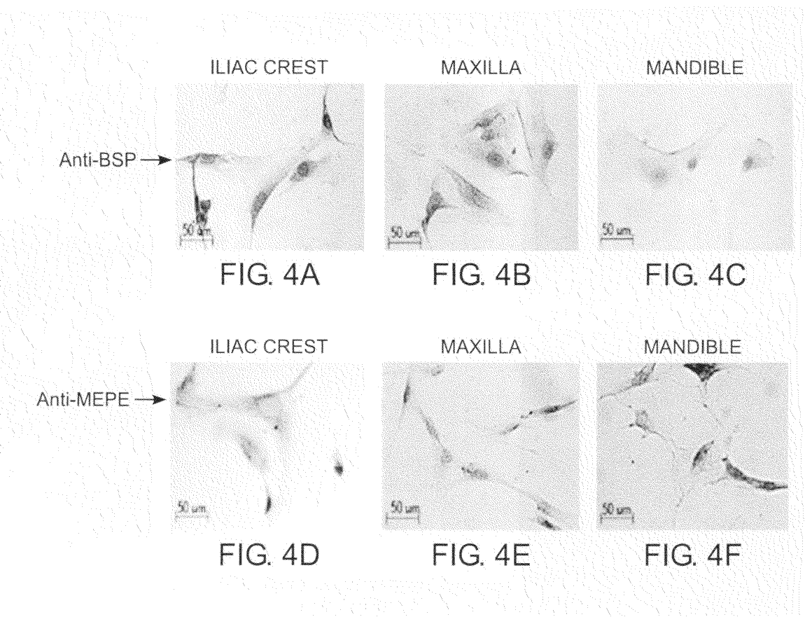

A panel of antibodies to 6 growth factors or matrix proteins identified some similarities and minor differences in hBMSC immunoreactivity (Table 1). Antibodies raised to human bone sialoprotein (BSP), matrix extracellular glycoprotein (MEPE), VEGF-R1 and FGF-R3 reacted with equal affinity in all three cell types, while antibodies to TGF-R2 and endostatin were less reactive with iliac crest hBMSCs (Table 1). A representative strong immunoreactivity of rabbit anti-human BSP (LF-120) and anti-human MEPE (LF 155) to hBMSCs from the three skeletal sites is shown in FIG. 4. Due to the higher immunoreactivity of TGFβ-R2 and endostatin to OF-MSCs compared with iliac crest hBMSCs, mRNA levels of TGFβ-R2 and endostatin were examined by semi-quantitative RT-PCR, but there were no significant differences between the cell types.

TABLE 1
Immunoreactivity of human bone marrow stromal
cells to six growth factors or matrix proteins

| Antibodies | Iliac crest | Maxilla | Mandible |
|---|---|---|---|
| BSP | ++ | ++ | ++ |
| MEPE | ++ | ++ | ++ |
| TGFβ-R2 | + | ++ | ++ |
| Endostatin | + | ++ | ++ |
| VEGF-R1 | + | + | + |
| bFGF-R3 | + | + | + |

Summary of comparative immunoreactivity of human bone marrow stromal cells (hBMSCs) to antibodies raised to 6 human cell surface markers. While immunostaining of maxilla and mandible hBMSCs was similar, iliac crest hBMSCs reacted with less affinity to anti-human TGFβ-R2 and anti-human endostatin (n = 4, mean of 3different experiments; + = immunoreactivity; ++ = moderate immunoreactivity; BSP = bone sialoprotein; MEPE = matrix extracellular phosphoglycoprotein; TGFβ-R2 = transforming growth factor receptor 2; bFGF-R3 = basic flbroblast growth factor receptor 3).

### Example 5

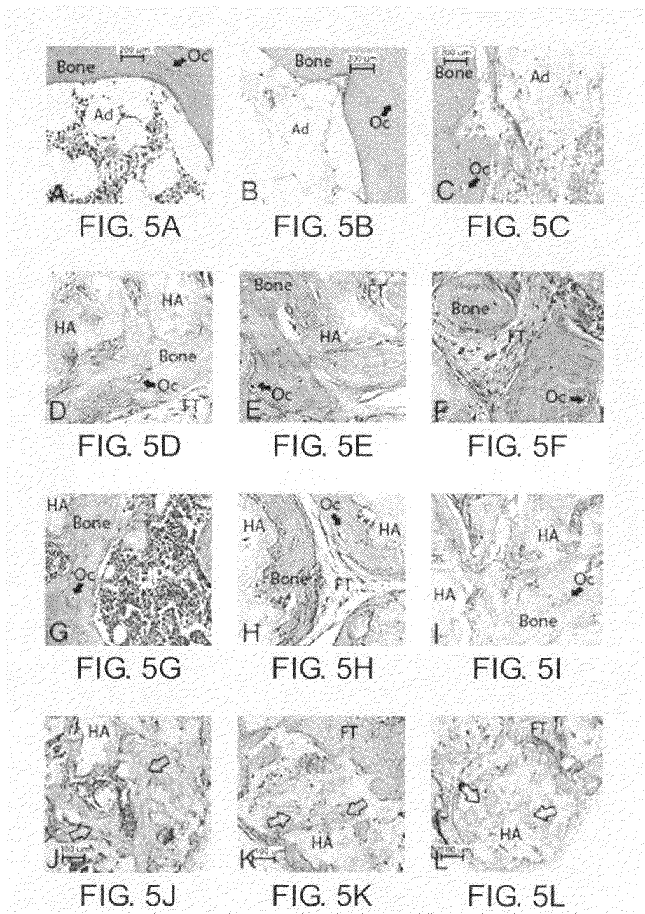

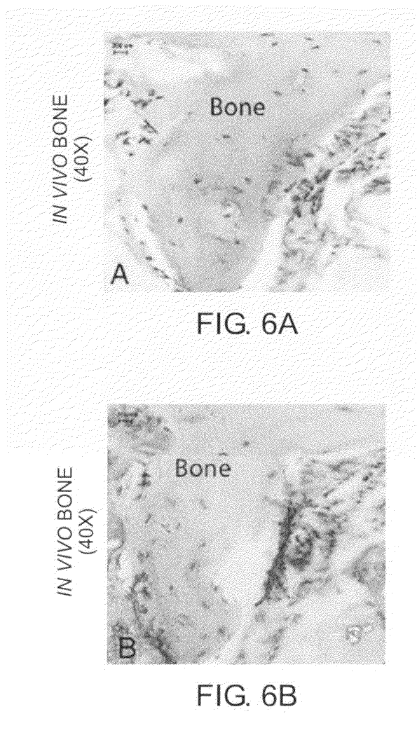

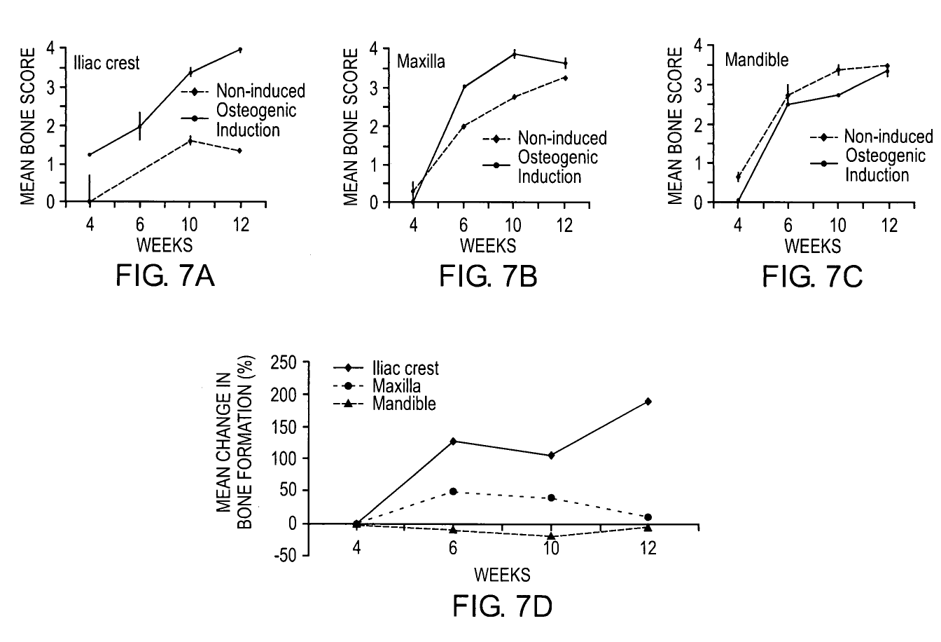

Evaluation of ectopic bone formation in nude mice clearly demonstrated that iliac crest hBMSCs formed a complete bone/marrow organ (FIG. 5). However, based on semi-quantitative bone scoring [25], iliac-crest-derived cells produced quantitatively less bone (FIG. 7) than orofacial cells. Morphologic comparisons of sections of normal human trabecular bone (FIGS. 5A, B, C) with new bone formed by non-induced (FIGS. 5D-5F) and osteogenically induced (FIGS. 5G-5I) hBMSCs transplanted into immunocompromised mice showed important differences. Dense bone with abundant adipose tissue was observed in sections of normal trabecular bone of iliac crest, maxilla and mandible (FIGS. 5A, 5B and 5C), but additional appreciable hematopoietic tissues were observed only in iliac crest (FIG. 5A). Interestingly, the histological pattern displayed by bone formed in vivo by transplanted hBMSCs was similar to that seen in normal bone only when the hBMSCs were induced to undergo osteogenesis in culture. Both non-induced (FIGS. 5D-5F) and osteogenically induced (FIGS. 5G-5I) hBMSCs from the three skeletal sites formed abundant bone, but hematopoiesis was evident only in bone formed by osteogenically induced iliac crest cells (FIG. 5G). Panoramic microscopic evaluation at low magnification (10×) showed bone formed by maxilla, and mandible cells were isolated nodules, unlike iliac crest cells that formed more closely packed bone with histologically observable hematopoietic marrow (FIGS. 5J-5L). Immunoreactivity with human-specific anti-osteopontin polyclonal antibody [6] demonstrated that bone formed within transplants from all three skeletal hBMSCs donor sites were of human origin (FIG. 6).

Further quantitative analysis of in vivo bone formation showed that OF-MSCs consistently formed more bone than iliac crest cells from weeks 4 to 12 (FIG. 7). Inter-observer agreement on bone score measurements was high based on a weighted Kappa statistic of 0.76. Unlike the in vitro osteogenic response displayed in FIG. 3, iliac crest cells pretreated with osteogenic inducer demonstrated more in vivo bone formation, with marked and sustained increase at each time point for 12 weeks (FIGS. 7A and D) compared to gradual rise from weeks 4-12 by maxilla (FIGS. 7B and D) and weak or negligible response by mandible cells (FIGS. 7C and D).

Osteogenic induction of iliac crest hBMSCs resulted in early response to in vivo bone formation (week 4) that was sustained for 12 weeks (FIG. 7A). In contrast, OF-MSCs did not respond differently to osteogenic stimulation at week 4. However, osteoinduced maxilla cells formed relatively more abundant bone in 12 weeks (FIG. 7B) than mandible cells (FIG. 7C).

The sustained rate of in vivo bone formation over 12 weeks by osteogenically induced iliac crest hBMSCs (FIG. 7A) is distinct from the rapid response or “catch-up” effect displayed by non-induced maxilla (FIG. 7B, at week 12) and mandible (FIG. 7C, weeks 4-12) OF-MSCs, which formed bone comparable to that formed by osteogenically induced cells. In addition, iliac crest hBMSCs formed more dense bone containing distinct marrow-like cavities (FIGS. 5G and J). These results indicate that in vivo response of hBMSCs to osteogenic induction is site-specific, and the iliac crest is most responsive while mandible is least responsive to induction (iliac crest>maxilla>mandible). Unlike iliac crest, it appears that OF-MSCs readily differentiated osteogenically in an in vivo model.

### Discussion of Experimental Examples

Starting with limited amounts of maxilla and mandible bone marrow samples from third molar surgical sites, hBMSCs were successfully isolated, expanded and partially characterized. OFMSCs displayed fibroblast-like morphology and formed isolated colonies in a manner similar to iliac crest hBMSCs. Both iliac crest hBMSCs and OF-MSCs contained STRO-1+ cells which identify a cell surface antigen expressed by stromal elements in human bone marrow. STRO-1, a monoclonal IgM produced from mouse immunized with CD34+ cells, recognizes only clonogenic and highly osteogenic progenitors. They share expression of CD34 with primitive hematopoietic precursors but are phenotypically distinct cell types. As these cells mature and differentiate, STRO-1 expression decreases while lineagespecific antigens increase [40].

Higher proliferation and osteogenic differentiation of OFMSCs without direct stimulation indicate that OF-MSCs do not need induction to differentiate osteogenically, whereas iliac crest hBMSCs responded as well as OF-MSCs both in vitro and in vivo after osteogenic stimulation. In addition, iliac crest hBMSCs were more responsive to adipogenic stimulation.

The similar in vitro and in vivo characteristics of OF-MSCs from both maxilla and mandible sampling sites compared with iliac crest underscore site-specific differences between orofacial and iliac crest hBMSCs. This may be a reflection of the amount of bone marrow progenitor cells as well as site-specific ontogeny. Although age influences quantity of resident BMSC population and osteogenic differentiation [29], the impact of age on tissue samples was minimal because hBMSCs from the three donor sites were compared in same individuals without cross-matching samples, the age range of subjects was within a narrow limit and below 35 years, the upper age limit of peak bone mass following which dramatic decrease in bone composition and mineral density occurs [11].

Differences in histological appearance of in vivo bone formed by the three cell types may relate to biochemical strain associated with functional demands at each skeletal site. OFMSCs formed isolated bone nodules, in contrast to the closely packed bone formed by iliac crest cells. Similarly, bone formed by OF-MSCs was separated by abundant fibrous tissue, unlike bone formed by iliac crest that contained appreciable hematopoietic components. These features are consistent with the different morphology of normal trabecular bone at the three sites and reflect site-specific functional demands. The ilium, a part of the pelvic girdle, is physiologically adapted for support of body weight, contains more red marrow and contributes more to hematopoiesis [5]. In contrast, maxilla and mandible are parts of the craniofacial complex, contain less red marrow but offer protection for vital structures such as air sinuses, dentition and neurovascular bundle.

It is plausible that observed skeletal site-specific differences of hBMSCs are related to different embryological origins and adaptation to functional demands at each skeletal site. Other extrinsic and intrinsic factors that may lead to observed differences include local vascular supply, the cellular composition and genetic profile of the marrow microenvironment, hormonal effects and muscular attachments that directly accentuate biochemical strains of mechanical load [8,23]. While some of the observed differences between OF-MSCs and appendicular skeleton hBMSC may be attributed to environmental factors, it seems less likely that the vastly more sustained population doublings of OF-MSCs in comparison to iliac crest cells are environmentally regulated.

The higher in vitro activity of the early osteogenic marker, alkaline phosphatase, by OF-MSCs (FIG. 3E) is consistent with the increased tendency of these cells to differentiate osteogenically in vivo without prior osteoinduction (FIG. 7). It will be important to determine if bone-associated transcription factors such as Runx2 (Cbfa1) and osterix, which promote osteoblast differentiation [7,28], are more highly expressed in the OF-MSCs. The more marked PD property of OF-MSCs despite less response to osteogenic stimulation indicates more self-renewal ability than iliac crest cells and a reflection of cells in an earlier stage of stromal cell differentiation. Since normal maxilla and mandible contain less hematopoietic marrow than ilium (FIGS. 6A, B and C) [31], the higher proliferation and PD are consistent with reports that stromal cells of nonhematopoietic marrow divide more actively unlike those of hematopoietic marrow that are usually mitotically quiescent while continuing to express the osteoblastic marker, alkaline phosphatase [2].

In view of the data set forth herein, maxilla and mandible are found to be important sources of hBMSCs for bone regeneration. Distinctive OF-MSCs phenotypes and differentiation support earlier, generic reports of the unique nature of craniofacial-specific multipotent stromal cells previously characterized from dental pulp [14], periodontal ligament [38], exfoliated deciduous teeth [27] and aspirate from dental extraction sockets [26]. The observation, set forth herein, that OF-MSCs need less induction than iliac crest hBMSCs to differentiate osteogenically in an in vivo model makes the maxilla and mandible prime donor sites for bone graft.

In summary, the present invention identifies fibroblast-like, colony forming and STRO 1+ hBMSCs in nucleated cells isolated from maxilla and mandible (orofacial) trabecular bone and compared them with those from iliac crest (axial bone) in same individuals. The findings set forth herein demonstrate the existence of skeletal sitespecific properties of orofacial and axial hBMSCs based on different embryological origins.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### ADDITIONAL REFERENCES

[1] Akintoye S O, Lee J S, Feimster T, Booher S, Brahim J, Kingman A, et al. Dental characteristics in fibrous dysplasia and McCune-Albright syndrome. Oral Surg Oral Med Oral Pathol Oral Radiol Endod 2003; 96:275-82.
[2] Bianco P, Riminucci M, Kuznetsov S, Robey P G. Multipotential cells in the bone marrow stroma: regulation in the context of organ physiology. Crit. Rev Eukaryot Gene Expr 1999; 9:159-73.
[3] Bradford M M. A rapid and sensitive method for the quantitation of microgram quantities of protein utilizing the principle of protein-dye binding. Anal Biochem 1976; 72:248-54.
[4] Chai Y, Jiang X, Ito Y, Bringas Jr P, Han J, Rowitch D H, et al. Fate of the mammalian cranial neural crest during tooth and mandibular morphogenesis. Development 2000; 127:1671-9.
[5] Charbord P, Tavian M, Humeau L, Peault B. Early ontogeny of the human marrow from long bones: an immunohistochemical study of hematopoiesisand its microenvironment. Blood 1996; 87:4109-19.
[6] Denhardt D T, Noda M. Osteopontin expression and function: role in bone remodeling. J Cell Biochem, Suppl 1998; 30-31:92-102.
[7] Ducy P, Zhang R, Geoffroy V, Ridall A L, Karsenty G. Osf2/Cbfa1: a transcriptional activator of osteoblast differentiation. Cell 1997; 89:747-54.
[8] Eriksen E F. Normal and pathological remodeling of human trabecularbone: three dimensional reconstruction of the remodeling sequence innormals and in metabolic bone disease. Endocr Rev 1986; 7:379-408.
[9] Fedarko N S, Bianco P, Vetter U, Robey P G. Human bone cell enzyme expression and cellular heterogeneity: correlation of alkaline phosphatase enzyme activity with cell cycle. J Cell Physiol 1990; 144:115-21.
[10] Fedarko N S, Avis D P, Frazier C R, Burrill M J, Fergusson V, Tayback M, et al. Cell proliferation of human fibroblasts and osteoblasts in osteogenesis imperfecta: influence of age. J Bone Miner Res 1995; 10:1705-12.
[11] Gordon C L, Halton J M, Atkinson S A, Webber C E. The contributions of growth and puberty to peak bone mass. Growth Dev Aging 1991; 55:257-62.
[12] Gronthos S, Brahim J, Li W, Fisher L W, Cheman N, Boyde A, et al. Stem cell properties of human dental pulp stem cells. J Dent Res 2002; 81:531-5.
[13] Gronthos S, Graves S E, Ohta S, Simmons P J. The STRO-1+ fraction of adult human bone marrow contains the osteogenic precursors. Blood 1994; 84:4164-73.
[14] Gronthos S, Mankani M, Brahim J, Robey P G, Shi S. Postnatal human dental pulp stem cells (DPSCs) in vitro and in vivo. Proc Natl Acad Sci USA 2000; 97:13625-30.
[15] Gronthos S, Zannettino A C, Hay S J, Shi S, Graves S E, Kortesidis A, et al. Molecular and cellular characterisation of highly purified stromal stem cells derived from human bone marrow. J Cell Sci 2003; 116:1827-35.
[16] Hayflick L, Moorhead P S. The serial cultivation of human diploid cell strains. Exp Cell Res 1961; 25:585-621.
[17] Helms J A, Schneider R A. Cranial skeletal biology. Nature 2003; 423:326-31.
[18] Jackson I T, Helden G, Marx R. Skull bone grafts in maxillofacial and craniofacial surgery. J Oral Maxillofac Surg 1986; 44:949-55.
[19] Kassem M, Ankersen L, Eriksen E F, Clark B F, Rattan S I. Demonstration of cellular aging and senescence in serially passaged long-term cultures of human trabecular osteoblasts. Osteoporos Int 1997; 7:514-24.
[20] Kuznetsov S, Gehron Robey P. Species differences in growth requirements for bone marrow stromal fibroblast colony formation In vitro. Calcif Tissue Int 1996; 59:265-70.
[21] Kuznetsov S A, Krebsbach P H, Satomura K, Kerr J, Riminucci M, Benayahu D, et al. Single-colony derived strains of human marrow stromal fibroblasts form bone after transplantation in vivo. J Bone Miner Res 1997; 12:1335-47.
[22] Leboy P S, Vaias L, Uschmann B, Golub E, Adams S L, Pacifici M. Ascorbic acid induces alkaline phosphatase, type X collagen, and calcium deposition in cultured chick chondrocytes. J Biol Chem 1989; 264:17281-6.
[23] Lips P, Courpron P, Meunier P J. Mean wall thickness of trabecular bone packets in the human iliac crest: changes with age. Calcif Tissue Res 1978; 26:13-7.
[24] Malpe R, Baylink D J, Linkhart T A, Wergedal J E, Mohan S. Insulin-like growth factor (IGF)-I, -II, IGF binding proteins (IGFBP)-3, -4, and -5 levels in the conditioned media of normal human bone cells are skeletal site-dependent. J Bone Miner Res 1997; 12:423-30.
[25] Mankani M H, Kuznetsov S A, Fowler B, Kingman A, Robey P G. In vivo bone formation by human bone marrow stromal cells: effect of carrier particle size and shape. Biotechnol Bioeng 2001; 72:96-107.
[26] Matsubara T, Suardita K, Ishii M, Sugiyama M, Igarashi A, Oda R, et al. Alveolar bone marrow as a cell source for regenerative medicine: differences between alveolar and iliac bone marrow stromal cells. J Bone Miner Res 2005; 20:399-409.
[27] Miura M, Gronthos S, Zhao M, Lu B, Fisher L W, Robey P G, et al. SHED: stem cells from human exfoliated deciduous teeth. Proc Natl Acad Sci USA 2003; 100:5807-12.
[28] Nakashima K, Zhou X, Kunkel G, Zhang Z, Deng J M, Behringer R R, et al. The novel zinc finger-containing transcription factor osterix is required for osteoblast differentiation and bone formation. Cell 2002; 108:17-29.
[29] Newton-John H F, Morgan D B. The loss of bone with age, osteoporosis, and fractures. Clin Orthop 1970; 71:229-52.
[30] Oklund S A, Prolo D J, Gutierrez R V, King S E. Quantitative comparisons of healing in cranial fresh autografts, frozen autografts and processed autografts, and allografts in canine skull defects. Clin Orthop 1986:269-91.
[31] Olsen B R, Reginato A M, Wang W. Bone development. Annu Rev Cell Dev Biol 2000; 16:191-220.
[32] Osyczka A M, Noth U, O'Connor J, Caterson E J, Yoon K, Danielson K G, et al. Multilineage differentiation of adult human bone marrow progenitor cells transduced with human papilloma virus type 16 E6/E7 genes. Calcif Tissue Int 2002; 71:447-58.
[33] Owen M, Friedenstein A J. Stromal stem cells: marrow-derived osteogenic precursors. Ciba Found Symp 1988; 136:42-60.
[34] Pereira R F, Halford K W, O'Hara M D, Leeper D B, Sokolov B P, Pollard M D, et al. Cultured adherent cells from marrow can serve as long-lasting precursor cells for bone, cartilage, and lung in irradiated mice. Proc Natl Acad Sci USA 1995; 92:4857-61.
[35] Riminucci M, Liu B. Corsi A, Shenker A, Spiegel A M, Robey P G, et al. The histopathology of fibrous dysplasia of bone in patients with activating mutations of the Gs alpha gene: site-specific patterns and recurrent histological hallmarks. J Pathol 1999; 187:249-58.
[36] Satomura K, Krebsbach P, Bianco P, Gehron Robey P. Osteogenic imprinting upstream of marrow stromal cell differentiation. J Cell Biochem 2000; 78:391-403.
[37] Sawin P D, Traynelis V C, Menezes A H. A comparative analysis of fusion S. O. Akintoye et al./Bone 38 (2006) 758-768 767 rates and donor-site morbidity for autogeneic rib and iliac crest bone grafts in posterior cervical fusions. J Neurosurg 1998; 88:255-65.
[38] Seo B M, Miura M, Gronthos S, Bartold P M, Batouli S, Brahim J, et al. Investigation of multipotent postnatal stem cells from human periodontal ligament. Lancet 2004; 364:149-55.
[39] Shi S, Gronthos S. Perivascular niche of postnatal mesenchymal stem cells in human bone marrow and dental pulp. J Bone Miner Res 2003; 18:696-704.
[40] Simmons P J, Torok-Storb B. Identification of stromal cell precursors inhuman bone marrow by a novel monoclonal antibody, STRO-1. Blood 1991; 78:5562.
[41] Simonds W F, James-Newton L A, Agarwal S K, Yang B, Skarulis M C, Hendy G N, et al. Familial isolated hyperparathyroidism: clinical and genetic characteristics of 36 kindreds. Medicine (Baltimore) 2002; 81:1-26.
[42] Ueki Y, Tiziani V, Santanna C, Fukai N, Maulik C, Garfinkle J, et al. Mutations in the gene encoding c-Abl-binding protein SH3BP2 cause cherubism. Nat Genet. 2001; 28:125-6.

What is claimed:

1. A method of detecting an orofacial-derived bone marrow stromal cell (OF-BMSC), said method comprising the steps of:
a. obtaining a first marrow stromal cell from a subject;
b. culturing said first marrow stomal cell;
c. comparing at least one property of said first marrow stromal cell with the corresponding characteristic of a second, non-orofacial marrow stromal cell;
wherein a difference between the characteristics of said first and second marrow stromal cells is an indication that the first cell is an OF-BMSC, said difference including at least one of the characteristics selected from the group consisting of positive presence of the STRO-1 marker, increased level of calcium accumulation, more rapid proliferation, delayed senescence, and higher expressed level of alkaline phosphatase, when compared to the characteristics of said second cell.

2. A method of enriching orofacial-derived bone marrow stromal cells (OF-BMSCs) from a population of bone marrow stromal cells containing at least one OF-BMSC, said method comprising:
a) providing an antibody specific for at least one marker expressed on an OF-BMSC;
b) contacting said population of cells with said antibody under conditions suitable for formation of an antibody-OF-BMSC complex;
c) substantially separating said antibody-OF-BMSC complex from said population of cells; and
d) identifying at least one OF-BMSC characteristic selected from the group consisting of increased level of calcium accumulation, rapid proliferation, delayed senescence, and higher expressed level of alkaline phosphatase;
thereby enriching said OF-BMSCs.

3. The method of claim 2, wherein said OF-BMSC is a human OF-BMSC.

4. The method of claim 2, wherein said marker is STRO-1, CD-106 and CD-146.

5. A method of providing an orofacial bone graft to a patient in need thereof, said method comprising:
a. detecting an orofacial marrow stromal cell according to the method of claim 1;
b. isolating said orofacial marrow stromal cell; and
c. administering said orofacial marrow stromal cell to said patient;
wherein said orofacial marrow stromal cell becomes engrafted to an orofacial bone in said patient.

6. A method of stimulating new bone formation in an orofacial bone in a patient in need thereof, said method comprising:
a. detecting an orofacial marrow stromal cell according to the method of claim 1;
b. isolating said orofacial marrow stromal cell; and
c. administering said orofacial marrow stromal cell to said patient;
wherein said orofacial marrow stromal cell stimulates new bone formation in an orofacial bone in said patient.

7. A method of providing an orofacial bone graft to a patient in need thereof, said method comprising administering an isolated orofacial marrow stromal cell to said patient, wherein said orofacial marrow stromal cell becomes engrafted to an orofacial bone in said patient.

8. A method of stimulating new bone formation in an orofacial bone in a patient in need thereof, said method comprising administering an isolated orofacial marrow stromal cell to said patient, wherein said orofacial marrow stromal cell stimulates new bone formation in an orofacial bone in said patient.
